(19) United States
(12) Patent Application Publication
(10) Pub. No.: US 2024/0270824 A1
(43) Pub. Date: Aug. 15, 2024

(54) HEMOGLOBIN (Hb) RECOMBINANT EXPRESSION VECTOR AND RECOMBINANT ENGINEERING BACTERIUM, AND PREPARATION METHOD AND USE THEREOF

(71) Applicants: Zhejiang University, Hangzhou City (CN); Ningbo Innovation Center, Zhejiang University, Ningbo City (CN); Shanghai Institute for Advanced Study of ZJU, Shanghai (CN)

(72) Inventors: Wei CHEN, Hangzhou City (CN); Lianghua XIE, Hangzhou City (CN); Yunhe MENG, Hangzhou City (CN); Kaihao YOU, Hangzhou City (CN)

(21) Appl. No.: 18/405,405

(22) Filed: Jan. 5, 2024

(30) Foreign Application Priority Data
Feb. 15, 2023 (CN) 202310117896.0

(51) Int. Cl. C07K 14/805; C12N 1/20; C12N 15/70; C12R 1/19

(52) U.S. Cl. CPC C07K 14/805; C12N 1/205; C12N 15/70; C12N 2500/34; C12R 2001/19

(57) ABSTRACT

The present disclosure provides a gene expression cassette encoding hemoglobin (Hb) and use thereof, and relates to the technical field of genetic engineering. In the present disclosure, a recombinant Escherichia coli strain with signal peptides Pel B, Pho A, and Omp A inserted under same conditions has an expression level of leghemoglobin (LegH) increased by 3.01, 1.25, and 1.22 times, respectively, compared with an original LegH expressing strain. The present disclosure provides signal peptides that can increase the expression level of the LegH, and provide a new idea for research and application of improving the expression level of the LegH.

### CROSS REFERENCE TO RELATED APPLICATION

This patent application claims the benefit and priority of Chinese Patent Application No. 202310117896.0 filed with the China National Intellectual Property Administration on Feb. 15, 2023, the disclosure of which is incorporated by reference herein in its entirety as part of the present application.

### REFERENCE TO SEQUENCE LISTING

A computer readable XML file entitled “GWP20231007898_Sequence_listing”, created on Nov. 23, 2023, with a file size of about 16,736 bytes, contains the sequence listing for this application, has been filed with this application, and is hereby incorporated by reference in its entirety.

### TECHNICAL FIELD

The present disclosure relates to the technical field of genetic engineering, and relates to a hemoglobin (Hb) recombinant expression vector and a recombinant engineering bacterium, and a preparation method and use thereof.

### BACKGROUND

Artificial meat is a type of edible meat produced without using traditional livestock and poultry breeding methods. The emergence of artificial meat provides new ideas for the current tense meat production and provides a solution to alleviate the imbalanced supply relationship of meat. As one of the main sensory indicators, color can directly affect consumers' purchasing and consumption desires. However, artificial meat products currently available cannot produce a color similar to real meat due to low heme content. To improve the appearance of artificial meat, some food additives are used in artificial meat to imitate the coloring of real meat. For example, exogenous addition of hemoglobin (Hb) is conducted to simulate the color of real meat.

Although the Hb can be obtained directly from plants or animals, this operation not only wastes time and energy, but also further increases the production cost of the artificial meat. Therefore, animal-free and biotechnological production of Hb is essential for high-yielding, economical, and environmental-friendly meat production. At present, there have been many reports on biosynthetic Hb research at home and abroad. This cost-effective production method has laid the foundation for the application of Hb in artificial meat. However, there are still many limitations in the biosynthesis of Hb, with a yield to be further improved.

Hemoglobin (Hb) is a ubiquitous iron-binding protein in nature and widely exists in microorganisms, animals, and plants. Hb present in plants is called plant Hb and was first discovered in the root nodules of legumes. In 1939, Kubo et al. found that the root nodules of legumes contained a red pigment similar to myohemerythrin of vertebrate horses, and may serve as a storage and carrier of oxygen. In 1945, the red pigment was further confirmed to be true Hb, which could form a completely reversible compound with oxygen molecules and had a high affinity for oxygen. Later, Hb present in legume root nodules was officially named leghemoglobin (LegH). The LegH is plant-derived Hb produced by the symbiotic system of rhizobia infected soybeans, and consists of a heme prosthetic group and a peptide chain. Compared with animal proteins, the amino acid sequence of LegH contains less histidine and no sulfur-containing amino acids at all. However, the above two types of proteins have highly-similar tertiary structures. LegH also releases heme cofactors during the cooking to catalyze the reaction of some biomolecules, thereby producing compounds with a meaty flavor. Therefore, the addition of LegH to artificial meat can greatly improve the color and flavor of the artificial meat, and has great application prospects.

At present, LegH is mainly extracted from soybeans. However, soybeans have a long planting cycle, and the soybeans have a relatively low yield natural LegH. The extraction process is complex, and the large-scale production costs extremely high. From the perspective of sustainable development, manufacturing Hb based on microbial culture technology to develop “artificial meat” solves the unsustainable problems of food raw materials and production methods, which is economical- and environmental-friendly, showing a great application potential and a scientific research value. In view of this, the production of LegH using microbial fermentation technology has attracted widespread attention and exhibits great market prospects.

### SUMMARY

Based on the deficiencies in the prior art, the present disclosure provides a hemoglobin (Hb) recombinant expression vector and a recombinant engineering bacterium, and a preparation method and use thereof. The efficient expression and secretion of LegH in Escherichia coli is achieved by introducing three signal peptides, Pel B, Pho A, and Omp A.

In the present disclosure, a signal peptide of pectin lyase Pel B from Erwinia carotovora, a signal peptide of alkaline phosphatase Pho A that regulates metabolism and transport of Escherichia coli, and a signal peptide of outer membrane protein Omp A of the Escherichia coli are inserted into a recombinant plasmid expressing LegH. The expression levels of LegH of the three signal peptides inserted recombinant plasmids expressing LegH are increased by 3.01, 1.25, and 1.22 times, respectively, compared with an original LegH expressing plasmid. The signal peptides better solve the problem of low LegH protein expression and lay the foundation for the biosynthesis of LegH and the simulation of artificial meat.

The present disclosure adopts the following technical solutions:

The present disclosure provides a gene expression cassette encoding Hb, including an encoding gene of a signal peptide and an encoding gene of soybean (Glycine max (Linn.) Merr.)-derived Hb sequentially; where the signal peptide is selected from the group consisting of a signal peptide Pel B, a signal peptide Pho A, and a signal peptide Omp A.

Specifically, the Hb has an encoding gene sequence set forth in SEQ ID NO: 4, the signal peptide Pel B has an encoding gene sequence set forth in SEQ ID NO: 1, the signal peptide Pho A has an encoding gene sequence set forth in SEQ ID NO: 2, and the signal peptide Omp A has an encoding gene sequence set forth in SEQ ID NO: 3.

The present disclosure further provides a plasmid including the gene expression cassette encoding Hb.

In an example of the present disclosure, the plasmid has an expression vector pET-28a.

A construction process of the plasmid includes the following steps:
subjecting a target gene of LegH and the pET-28a plasmid to double digestion by BamH I and Xho I endonucleases, ligating to obtain a pET-28a-LegH expression plasmid, and sequencing to verify a correctness of the target gene;
extracting an exogenous genome of an Escherichia coli TOP10 strain, amplifying Pel B, Pho A, and Omp A genes by PCP using a primer signal-F and a primer LegH-signal-R with a pUC57 genome as a template, respectively; and
subjecting the pET-28a-LegH expression plasmid to double digestion with Xba I and Nde I, purifying, ligating the signal peptide genes and the vector with a homologous recombinase to obtain recombinant plasmids pET-28a-Pel B-LegH, pET-28a-Pho A-LegH, and pET-28a-Omp A-LegH that have the Pel B, Pho A, and Omp A signal peptides, respectively, and conducting colony PCR and sequencing to verify the recombinant plasmids.

The present disclosure further provides a cell including the plasmid.

In an example of the present disclosure, the cell is an Escherichia coli BL21 (DE3) cell.

The present disclosure further provides use of the cell in preparation of Hb.

The present disclosure further provides a protein expression method, including the following steps: inoculating the cell into an LB medium to allow culture until an OD₆₀₀ value is 0.6, adding isopropyl-β-d-thiogalactoside (IPTG) with a final concentration of 0.01 mM to 1 mM, and conducting induction at 16° C. to 37° C. and 180 rpm for 8 h to 20 h.

Preferably, the IPTG at a final concentration of 1 mM is added to allow the induction at 25° C. and 180 rpm for 12 h.

LegH engineering strains expressing different signal peptides are cultured separately, and then subjected to induced expression according to the above conditions to screen and express the engineering strains, and a protein expression level is detected by SDS-PAGE.

The present disclosure has following beneficial effects:
1. In the present disclosure, recombinant Escherichia coli strains with signal peptides Pel B, Pho A, and Omp A inserted under same conditions have an expression level of LegH increased by 3.01, 1.25, and 1.22 times, respectively, compared with an original LegH expressing strain.
2. The present disclosure provides signal peptides that can increase the expression level of the LegH, and provides a new idea for research and application of improving the expression level of the LegH.

### BRIEF DESCRIPTION OF THE DRAWINGS

FIG. 1 shows a plasmid map of pET-28a-Pel B-LegH.

FIG. 2 shows a plasmid map of pET-28a-Pho A-LegH.

FIG. 3 shows a plasmid map of pET-28a-Omp A-LegH.

FIG. 4 is a figure showing verification results of fragment sizes of the three signal peptides Pel B, Pho A, and Omp A; where Lane 1: 100 bp DNA Ladder, Lanes 2 and 3: Pel B amplified fragments, Lanes 4 and 5: Pho A amplified fragments, Lanes 6 and 7: Omp A amplified fragments.

FIG. 5 is a figure showing verification results of plate colony PCR using pET-28a-Pel B-LegH recombinant plasmid to transform BL21 (DE3); where Lane 1: 100 bp DNA Ladder; Lanes 2 to 11: verification results of plate colony PCR transformed with pET-28a-Pel B-LegH.

FIG. 6 is a figure showing verification results of plate colony PCR using pET-28a-Pho A-LegH recombinant plasmid to transform BL21 (DE3); where Lane 1: 100 bp DNA Ladder; Lanes 2 to 11: verification results of plate colony PCR transformed with pET-28a-Pho A-LegH.

FIG. 7 is a figure showing verification results of plate colony PCR using pET-28a-Omp A-LegH recombinant plasmid to transform BL21 (DE3); where Lane 1: 100 bp DNA Ladder; Lanes 2 to 11: verification results of plate colony PCR transformed with pET-28a-Omp A-LegH.

FIG. 8 is a figure showing induced expression results of LegH with different signal peptides; an expression plasmid of LegH protein with signal peptide is transformed into BL21 (DE3) competent cells, and IPTG with a final concentration of 1 mM is added at OD₆₀₀=0.6 to induce the protein expression for 12 h; where Lane 1: protein marker; Lane 2: LegH protein expression without signal peptide; Lane 3: LegH protein expression with Pel B signal peptide; Lane 4: LegH protein expression with Pho A signal peptide; Lane 5: LegH protein expression with signal peptide Omp A;

FIG. 9 is a figure showing relative expression levels of LegH in strains with different signal peptides; where ** represents p<0.01, **** represents p<0.0001.

FIG. 10 is a figure showing an influence of IPTG with different concentrations on an expression level of the target protein; where Lane 1: protein marker; Lane 2: 0.01 mM IPTG induced LegH protein expression; Lane 3: 0.02 mM IPTG induced LegH protein expression; Lane 4: 0.05 mM IPTG induced LegH protein expression; Lane 5: 0.1 mM IPTG induced LegH protein expression; Lane 6: 0.2 mM IPTG induced LegH protein expression; Lane 7: 0.5 mM IPTG induced LegH protein expression; Lane 8: 1 mM IPTG induced LegH protein expression.

FIG. 11 is a figure showing an influence of different induction temperatures on the expression level of the target protein; where Lane 1: protein marker; Lane 2: LegH protein expression induced at 16° C.; Lane 3: LegH protein expression induced at 20° C.; Lane 4: LegH protein expression induced at 25° C.; Lane 5: LegH protein expression induced at 30° C.; Lane 6: LegH protein expression induced at 34° C.; Lane 7: LegH protein expression induced at 37° C.

FIG. 12 is a figure showing an influence of different induction times on the expression level of the target protein; where Lane 1: protein marker; Lane 2: LegH protein expression induced for 8 h; Lane 3: LegH protein expression induced for 12 h; Lane 4: LegH protein expression induced for 16 h; Lane 5: LegH protein expression induced for 20 h.

### DETAILED DESCRIPTION OF THE EMBODIMENTS

LB liquid medium: 10 g/LTryptone, 5 g/L Yeast Extract, 5 g/L NaCl, sterilized at 121° C. for 20 min, for the culturing of Escherichia coli.

LB solid medium: LB liquid medium added with 20 g/L agar powder, for the culturing and screening of Escherichia coli.

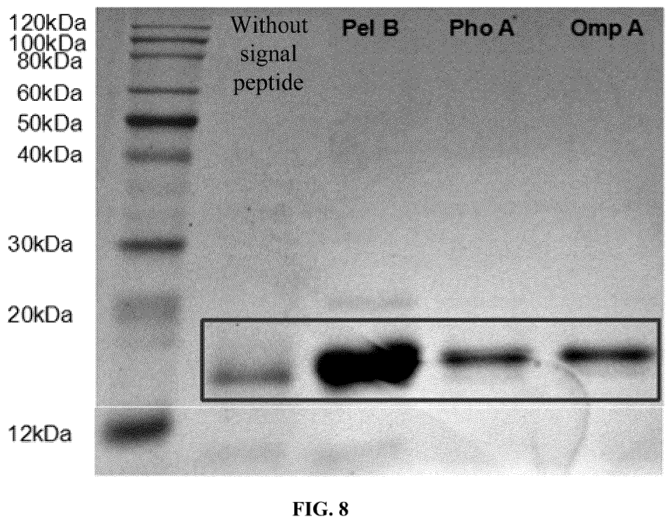

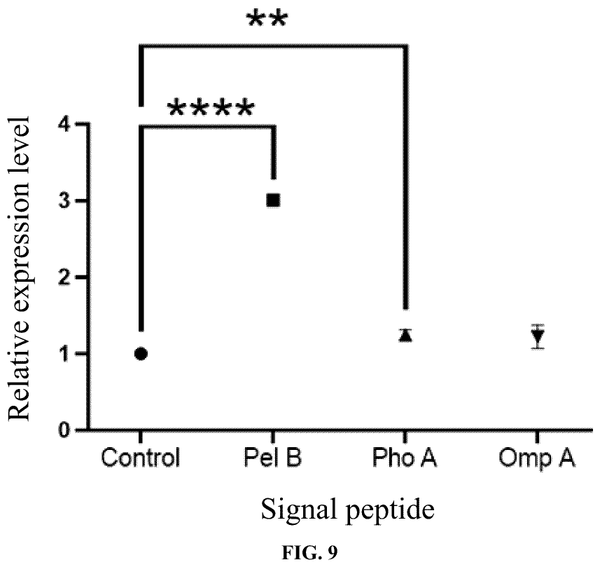

In the present disclosure, the LegH proteins with different signal peptides appear as bands at expected positions separately, and a molecular weight of the protein is consistent with a theoretical value. FIG. 8 shows a comparison of the expression levels of LegH proteins with different signal peptides under the same induction conditions; FIG. 9 shows the relative expression levels of LegH proteins with different signal peptides. Specific situations and results are given in the following examples.

### Example 1

1. Construction of pET-28a-LegH Vector

A LegH gene with gene sequence obtained from NCBI (a gene sequence was set forth in SEQ ID NO: 4) was synthesized by Tsingke Biotech Co., Ltd. And the target gene was amplified by PCR using primers LegH-F and LegH-R in Table 2. A pET-28a empty vector was double-digested using BamH I and Xho I enzymes (Takara, Dalian), and then the synthesized gene was ligated to the digested pET-28a vector using a homologous recombinase Trelief® Seamless Cloning Kit (Tsingke Biotech, Hangzhou) to construct a recombinant expression vector pET-28a-LegH of LegH.

### 2. Construction of Recombinant Plasmid Expression Vector

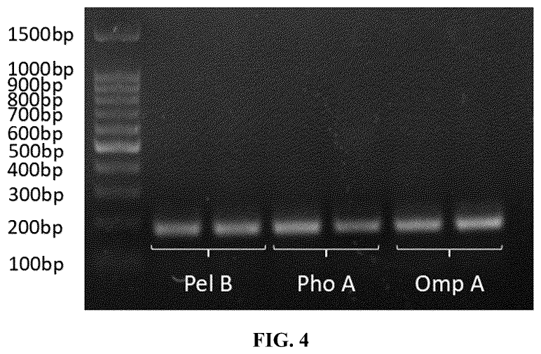

Step S1: an exogenous genome of E. coli Top10 strain (Tsingke Biotech, Hangzhou) was extracted using Yeasen kit; PCR was conducted using a pUC57 genome as a template and primers signal-F and LegH-signal-R in Table 2 to amplify genes encoding Pel B, Pho A, and Omp A; a size of the target gene fragment was identified by agarose gel electrophoresis (FIG. 4), and amino acid sequences and gene sequences encoding the signal peptides Pel B, Pho A, and Omp A were shown in Table 1.

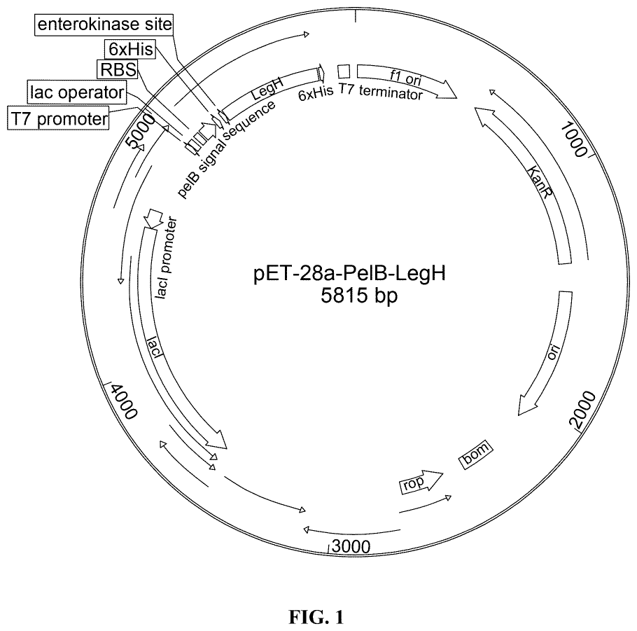

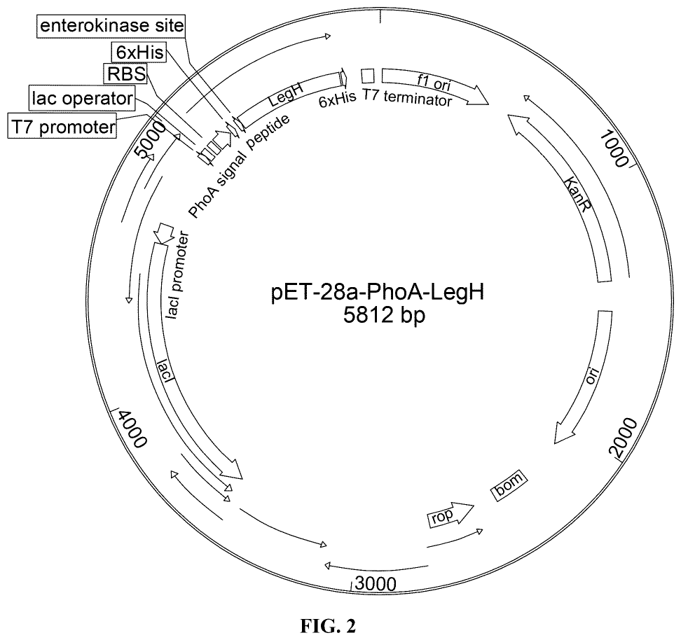

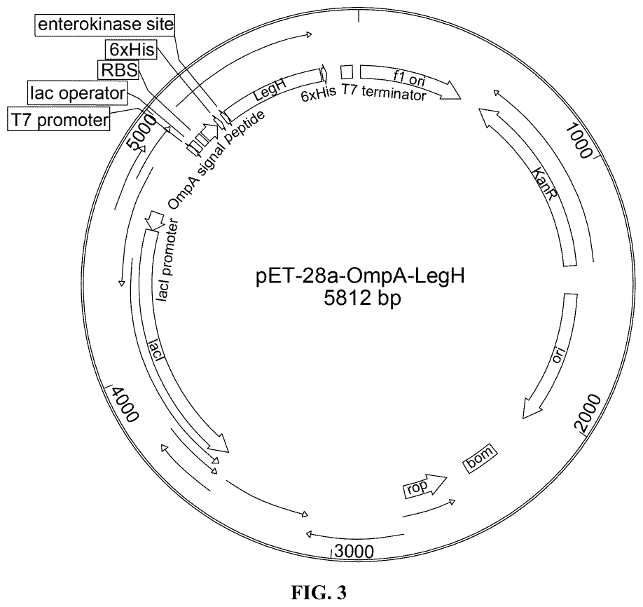

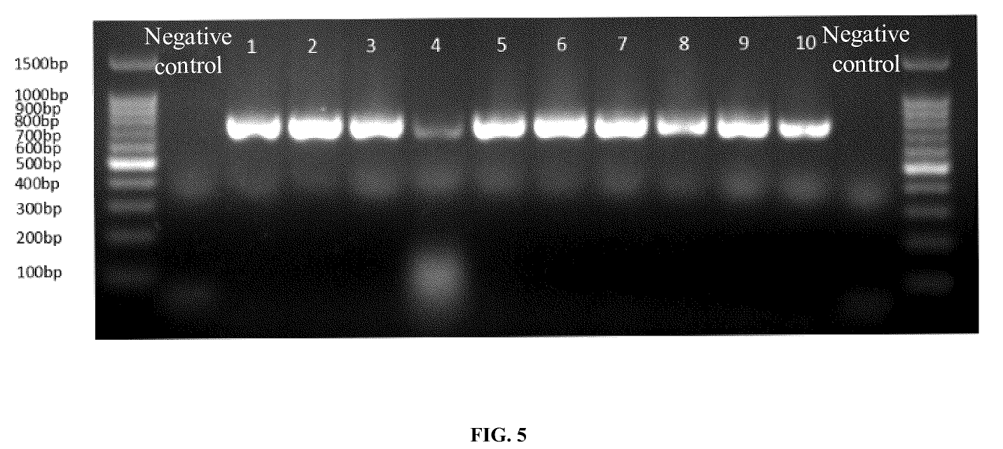

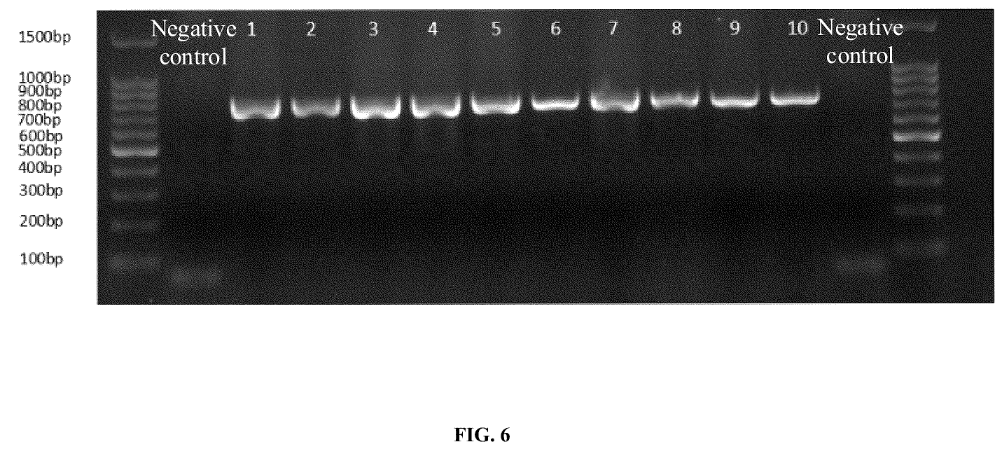

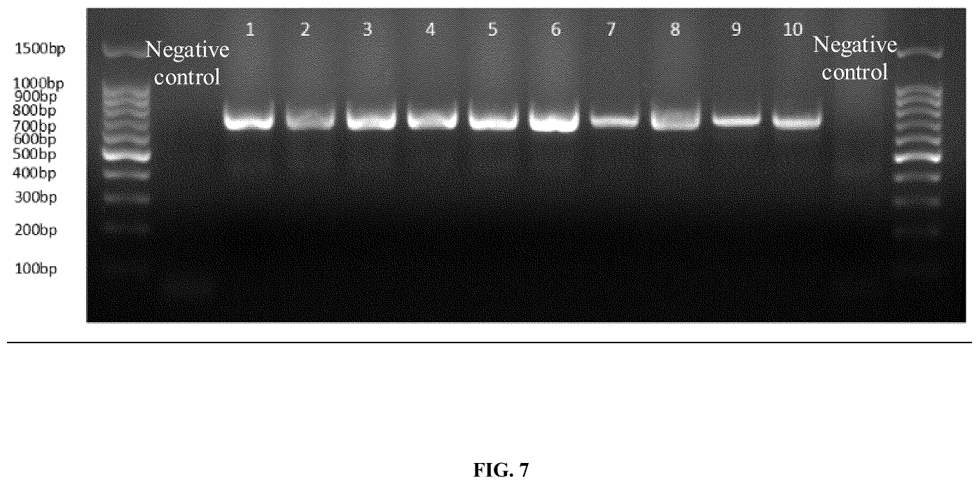

Step S2: the pET-28a-LegH plasmid was double-digested using Xba I and Nde I enzymes, and then purified; the signal peptide encoding genes (encoding Pel B, Pho A, and Omp A genes) obtained in step S1 and the digested vector pET-28a-LegH were ligated with a homologous recombinase Trelief® Seamless Cloning Kit, to obtain pET-28a-Pel B-LegH (with a map shown in FIG. 1), pET-28a-Pho A-LegH (with a map shown in FIG. 2), and pET-28a-Omp A-LegH plasmid (with a map shown in FIG. 3) containing gene sequences encoding the signal peptides Pel B, Pho A, and Omp A, respectively; the plasmids were transformed into E. coli BL21 (DE3) competent cells, spread on a LB solid medium containing 1%% of 50 μg/mL kanamycin, and then incubated inverted overnight at 37° C. 10 single colonies on each plate were put in 10 μL sterilized water, colony PCR and sequencing were conducted separately using obtained bacterial solutions of the 10 single clones as a template, T7-F and T7ter-R in Table 2 as upstream primer and downstream primer to verify the constructed recombinant plasmid (the negative control used sterilized water as a template). The colony PCR verification results were shown in FIG. 5 to FIG. 7. The single colonies selected from the transformation plates of the three recombinant plasmids containing signal peptides all showed bands at the expected positions, while the negative control had no bands at the corresponding positions. This indicated that the three recombinant plasmids containing signal peptides were successfully constructed and transformed into the E. coli BL21(DE3) competent cells.

TABLE 1
Amino acid and base sequences of different signal peptides

| Name | kDa | Amino acid sequence | Nucleotide sequence |
|---|---|---|---|
| Pel B | 2.23 | MKYLLPTAAAGLLL | 5′-ATGAAATACCTGCTGCCGACCGC |
| | | LAAQPAMA (SEQ ID | TGCTGCTGGTCTGCTGCTCCTCGCT |
| | | NO: 5) | GCCCAGCCGGCGATGGCC-3′ (SEQ |
| | | | ID NO: 1) |
| Pho A | 2.26 | MKQSTIALALLPLLF | 5′-ATGAAGCAGAGCACCATCGCCC |
| | | TPVTKA (SEQ ID NO: | TGGCCCTGCTGCCCCTGCTGTTCA |
| | | 6) | CCCCCGTGACCAAGGCC-3′ (SEQ ID |
| | | | NO: 2) |
| Omp A | 2.05 | MKKTAIAIAVALAGF | 5′-ATGAAGAAGACCGCCATCGCCA |
| | | ATVAQA (SEQ ID NO: | TCGCCGTGGCCCTGGCCGGCTTCG |
| | | 7) | CCACCGTGGCCCAGGCC-3′ (SEQ ID |
| | | | NO: 3) |

TABLE 2
Primer sequences for constructing vectors

| | | Restriction site | |
|---|---|---|---|
| Primer name | Primer sequence | (underlined part) | Use |
| LegH-F | 5′-TGGACAGCAAATGGGTCGCGGATCCATGG | BamHI | Upstream primer |
| | GTGCTTTCACTGAGAA-3′ (SEQ ID NO: 9) | | for LegH |
| | | | amplification |
| LegH-R | 5′-AGTGGTGGTGGTGGTGGTGCTCGAGTTAA | Xho I | Downstream |
| | GGTTCCATACAGCAGC-3′ (SEQ ID NO: 10) | | primer for LegH |
| | | | amplification |
| signal-F | 5′-GAGCGGATAACAATTCCCCTCTAGAAATAA | Xba I | Upstream primers |
| | TTTTGTTTAACTTTA-3′ (SEQ ID NO: 11) | | for amplification |
| | | | of three signal |
| | | | peptides |
| LegH-signal- | 5′-ATCCCTTGTCGTCGTCGTCCATATGAGAAC | Nde I | Downstream |
| R | CAGAACCATGATGAT-3′ (SEQ ID NO: 12) | | primers for |
| | | | amplification of |
| | | | three signal |
| | | | peptides |
| T7-F | 5′-TAATACGACTCACTATAGGG-3′ (SEQ ID | — | Upstream primers |
| | NO: 13) | | for colony PCR |
| | | | validation |
| T7ter-R | 5′-TGCTAGTTATTGCTCAGCGG-3′ (SEQ ID | — | Downstream |
| | NO: 14) | | primers for colony |
| | | | PCR validation |

### Example 2

### 1. Optimization of Induced Expression Conditions

1.1 Influence of IPTG with Different Concentrations on the Expression Level of Target Protein

The LegH target gene sequence was ligated to the pET-28a vector. For specific steps, see step 1 in Example 1. The plasmid was transformed into BL21 (DE3) competent cells for expression to determine the induced expression conditions of LegH. A single colony of E. coli that was successfully transformed and sequenced correctly was inoculated into a test tube with 5 mL kanamycin-containing liquid LB, and then cultured overnight at 37° C. and 220 rpm. A resulting bacterial solution cultured overnight was inoculated into 100 mL of kanamycin-resistant LB liquid medium at an inoculum volume of 1%, cultured at 37° C. and 220 rpm, and an OD value was detected. At a time point when the OD₆₀₀ value was 0.6, IPTG with final concentrations of 0.01 mM, 0.02 mM, 0.05 mM, 0.1 mM, 0.2 mM, 0.5 mM, and 1 mM were added separately to induce for 20 h at 20° C. and 180 rpm. SDS-PAGE was conducted to compare the influence of IPTG with different concentrations on the expression level of the target protein (the results were shown in FIG. 10), thereby determining an optimal IPTG concentration.

### 1.2 Influence of Different Induction Temperatures on the Expression Level of Target Protein

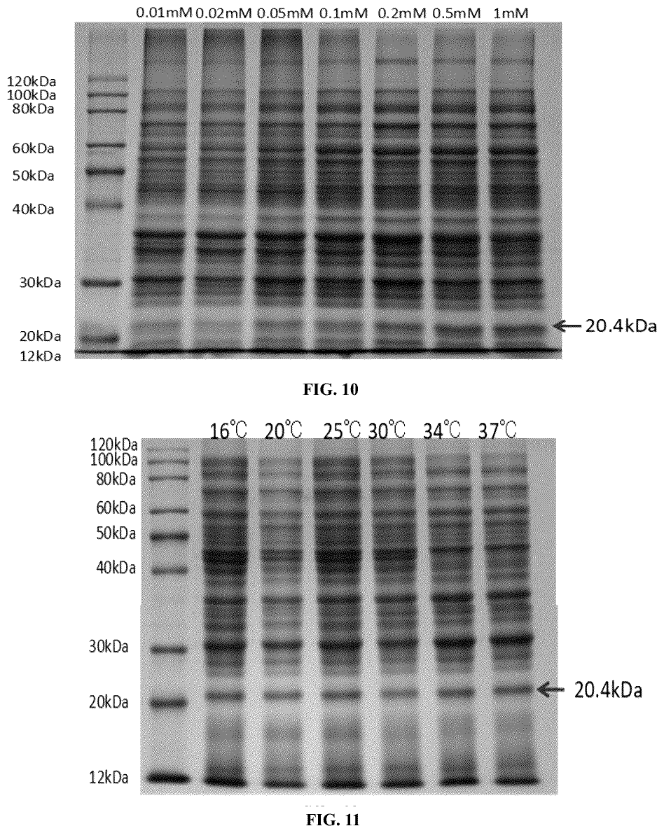

An induction temperature was adjusted according to the optimal IPTG concentration. A single colony of E. coli that was successfully transformed and sequenced correctly was inoculated into a test tube with 5 mL kanamycin-containing liquid LB, and then cultured overnight at 37° C. and 220 rpm. A resulting bacterial solution cultured overnight was inoculated into 100 mL of kanamycin-resistant LB liquid medium at an inoculum volume of 1%, cultured at 37° C. and 220 rpm, and an OD value was detected. At a time point when the OD₆₀₀ value was 0.6, an appropriate concentration of IPTG was added to induce at 16° C., 20° C., 25° C., 30° C., 34° C., and 37° C. and 180 rpm for 20 h. SDS-PAGE was conducted to compare the influence of different induction temperatures on the expression level of the target protein (the results were shown in FIG. 11), thereby determining an optimal induction temperature.

### 1.3 Influence of Different Induction Times on the Expression Level of Target Protein

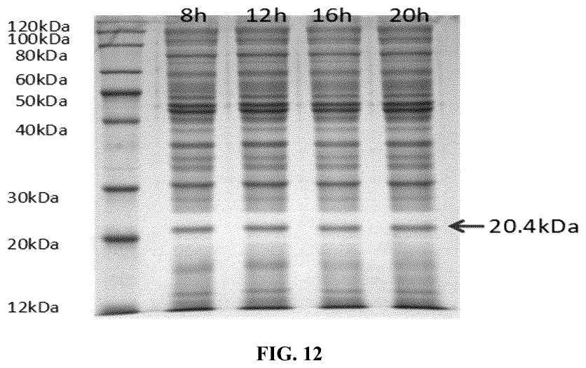

An induction time was adjusted according to the optimal IPTG concentration and optimal induction temperature. A single colony of E. coli that was successfully transformed and sequenced correctly was inoculated into a test tube with 5 mL kanamycin-containing liquid LB, and then cultured overnight at 37° C. and 220 rpm. A resulting bacterial solution cultured overnight was inoculated into 100 mL of kanamycin-resistant LB liquid medium at an inoculum volume of 1%, cultured at 37° C. and 220 rpm, and an OD value was detected. At a time point when the OD₆₀₀ value was 0.6, an appropriate concentration of IPTG was added to induce at 25° C. and 180 rpm for 8 h, 12 h, 16 h, and 20 h. SDS-PAGE was conducted to compare the influence of different induction times on the expression level of the target protein (the results were shown in FIG. 12), thereby determining an optimal induction time.

According to the optimization results of induced expression, the expression conditions of LegH protein without signal peptide were optimized. Under the condition of OD₆₀₀=0.6, the best induction conditions were obtained by adding IPTG with a final concentration of 1 mM and inducing at 25° C. and 180 rpm for 12 h.

2. Induced Expression of LegH Protein Containing Signal Peptide in E. coli

The expression vectors pET-28a-Pel B-LegH, pET-28a-Pho A-LegH, and pET-28a-Omp A-LegH constructed containing signal peptides in Example 1 were transformed into DH5α. E. coli competent cells, respectively. Transformants were selected for sequencing verification, and plasmid extraction was conducted after successful verification. The three LegH protein expression plasmids with different signal peptides were transformed into BL21 (DE3) E. coli competent cells, and the transformants were selected for sequencing verification. After successful verification, induced expression was conducted. Transformation system: 5 μL of recombinant plasmid and 100 μL of competent cells. A resulting mixed system was placed on ice for 30 min, heat-shocked at 42° C. for 80 s. After the heat shock was completed, the mixed system was placed on ice for 2 min, and then added with 700 μL of LB medium without resistance, cultured at 37° C. for 1 h, then centrifuged at 4,000 rpm for 3 min. The supernatant was discarded, and the pellet was resuspended in 200 μL of sterilized water, then spread on LB solid medium containing 1%% of 50 μg/mL kanamycin and incubated inverted overnight at 37° C. The next day, single clone transformants were picked for verification.

Induced expression: induced expression was conducted according to the optimization results of induced expression in step 1.

The correctly sequenced monoclonal bacterial solution was inoculated into 5 mL LB liquid medium containing 1%% of 50 μg/mL kanamycin, and cultured overnight at 37° C. and 220 rpm. The bacterial solution cultured overnight was inoculated into 100 mL liquid LB medium containing 1%% of 50 μg/mL kanamycin at an inoculum volume of 1%, and cultured at 37° C. and 220 rpm until OD₆₀₀=0.6. The IPTG with a final concentration 0.01 mM was added, and low-temperature induction was conducted at 20° C. and 180 rpm for 12 h.

### 3. His-Tag Protein Purification

Preliminary purification was conducted on the protein in the induction supernatant using HisTALON™ Gravity Column.

The induced bacterial solution was centrifuged at 4° C. and 4,000 rpm for 30 min, and a supernatant was discarded. The centrifuged pellet was resuspended in PBS and then disrupted by sonication. The disrupted sample was centrifuged at 4° C. and 4,000 rpm for 30 min, and a resulting supernatant was passed through a 0.45 μm aqueous filter membrane. The protein in the induction supernatant was subjected to preliminary column purification using the purification steps of HisTALON™ Gravity Column.

Protein expression levels were identified by SDS-PAGE, and relative quantification of the protein expression was conducted using Image J. The target protein was quantified using the Shenhua(SH) gel imaging system, and the relative expression level of LegH without signal peptide was set as 1.00. Accordingly, the relative expression level of LegH with signal peptide Pel B was 3.01, the relative expression level of LegH with signal peptide Pho A was 1.25, and the relative expression level of LegH with signal peptide Omp A was 1.22 (FIG. 8 and FIG. 9). These results showed that among the three signal peptides of the present disclosure, all signal peptides could increase the expression level of LegH to varying degrees, and the increased protein expression levels from high to low were: Pel B>Pho A>Omp A.

In the present disclosure, after the signal peptide Pel B was inserted into the LegH recombinant expression vector, the expression level of LegH was increased by 3.01 times compared with that without the signal peptide; after the signal peptide Pho A was inserted into the LegH recombinant expression vector, the expression level of LegH was increased by 1.25 times compared with that without the signal peptide; after the signal peptide Omp A was inserted into the LegH recombinant expression vector, the expression level of LegH was increased by 1.22 times compared with that without the signal peptide.

What is claimed is:

1. A gene expression cassette encoding hemoglobin (Hb) comprising: an encoding gene of a signal peptide and an encoding gene of soybean (Glycine max (Linn.) Merr.)-derived Hb sequentially; wherein the signal peptide is selected from the group consisting of a signal peptide Pel B, a signal peptide Pho A, and a signal peptide Omp A.

2. The gene expression cassette encoding Hb according to claim 1, wherein the gene sequence encoding Hb is set forth in SEQ ID NO: 4, the gene sequence encoding signal peptide Pel B is set forth in SEQ ID NO: 1, the gene sequence encoding signal peptide Pho A is set in SEQ ID NO: 2, and the gene sequence encoding signal peptide Omp A is set forth in SEQ ID NO: 3.

3. A cell comprising a plasmid, wherein the plasmid comprises the gene expression cassette encoding Hb according to claim 1.

4. The cell according to claim 3, wherein the cell is an Escherichia coli BL21 (DE3) cell.

5. A protein expression method, comprising the following steps: inoculating the cell according to claim 3 into an LB medium to allow culture until an OD₆₀₀ value is 0.6, adding isopropyl-β-d-thiogalactoside (IPTG) with a final concentration of 0.01 mM to 1 mM, and conducting induction at 16° C. to 37° C. and 180 rpm for 8 h to 20 h.

6. The protein expression method according to claim 5, wherein the IPTG at a final concentration of 1 mM is added to allow the induction at 25° C. and 180 rpm for 12 h.

7. The cell according to claim 3, wherein the gene sequence encoding Hb is set forth in SEQ ID NO: 4, the gene sequence encoding signal peptide Pel B is set forth in SEQ ID NO: 1, the gene sequence encoding signal peptide Pho A is set in SEQ ID NO: 2, and the gene sequence encoding signal peptide Omp A is set forth in SEQ ID NO: 3.

8. The cell according to claim 3, wherein the plasmid further comprises an expression vector pET-28a.


SEQUENCE LISTING (14 sequences)

SEQ ID NO: 1 - DNA, 66 bp, synthetic construct
  source 1..66: mol_type = other DNA; note = Gene sequence of Pel B from Erwinia carotovora
atgaaatacctgctgccgaccgctgctgctggtctgctgctcctcgctgcccagccggcgatggcc

SEQ ID NO: 2 - DNA, 63 bp, synthetic construct
  source 1..63: mol_type = other DNA; note = Gene sequence of Pho A from Escherichia coli Top10
atgaagcagagcaccatcgccctggccctgctgcccctgctgttcacccccgtgaccaaggcc

SEQ ID NO: 3 - DNA, 63 bp, synthetic construct
  source 1..63: mol_type = other DNA; note = Gene sequence of Omp A from Escherichia coli Top10
atgaagaagaccgccatcgccatcgccgtggccctggccggcttcgccaccgtggcccaggcc

SEQ ID NO: 4 - DNA, 444 bp, synthetic construct
  source 1..444: mol_type = other DNA; note = Gene sequence of Hb from Glycine max (Linn.) Merr.
atgggtgctttcactgagaagcaagaagccctcgtctcaagctcctttgaagcgttcaaagccaacattccgcagtatagcgttgtgttttatacctctattctggaaaaggccccagctgcaaaagatctgtttagctttttgtcgaacggggtagaccccagcaatccgaaactgaccggtcatgcagaaaaacttttcggcctggttcgtgatagcgcgggtcagctgaaggccaacgggacagtcgtcgcagatgcagcattgggatcgatccacgcgcagaaagccattaccgatccacaattcgtggtcgtaaaggaggcattattaaagacgataaaagaggcagttggtgataaatggtcagacgaactatctagtgcatgggaagttgcttacgacgagctagcggctgctatcaaaaaagcattttttctgaat

SEQ ID NO: 5 - protein, 22 aa, synthetic construct
  source 1..22: note = Amino acid sequence of Pel B from Erwinia carotovora
MKYLLPTAAAGLLLLAAQPAMA

SEQ ID NO: 6 - protein, 21 aa, synthetic construct
  source 1..21: note = Amino acid sequence of Pho A from Escherichia coli Top10
MKQSTIALALLPLLFTPVTKA

SEQ ID NO: 7 - protein, 21 aa, synthetic construct
  source 1..21: note = Amino acid sequence of Omp A from Escherichia coli Top10
MKKTAIAIAVALAGFATVAQA

SEQ ID NO: 8 - protein, 148 aa, synthetic construct
  source 1..148: note = Glycine max (Linn.) Merr.
MGAFTEKQEALVSSSFEAFKANIPQYSVVFYTSILEKAPAAKDLFSFLSNGVDPSNPKLTGHAEKLFGLVRDSAGQLKANGTVVADAALGSIHAQKAITDPQFVVVKEALLKTIKEAVGDKWSDELSSAWEVAYDELAAAIKKAFFLN

SEQ ID NO: 9 - DNA, 45 bp, synthetic construct
  source 1..45: mol_type = other DNA; note = Primer LegH-F
tggacagcaaatgggtcgcggatccatgggtgctttcactgagaa

SEQ ID NO: 10 - DNA, 45 bp, synthetic construct
  source 1..45: mol_type = other DNA; note = Primer LegH-R
agtggtggtggtggtggtgctcgagttaaggttccatacagcagc

SEQ ID NO: 11 - DNA, 45 bp, synthetic construct
  source 1..45: mol_type = other DNA; note = Primer signal-F
gagcggataacaattcccctctagaaataattttgtttaacttta

SEQ ID NO: 12 - DNA, 45 bp, synthetic construct
  source 1..45: mol_type = other DNA; note = Primer LegH-signal-R
atcccttgtcgtcgtcgtccatatgagaaccagaaccatgatgat

SEQ ID NO: 13 - DNA, 20 bp, synthetic construct
  source 1..20: mol_type = other DNA; note = Primer T7-F
taatacgactcactataggg

SEQ ID NO: 14 - DNA, 20 bp, synthetic construct
  source 1..20: mol_type = other DNA; note = Primer T7ter-R
tgctagttattgctcagcgg